(19) United States
(12) Patent Application Publication
(10) Pub. No.: US 2004/0082614 A1
(43) Pub. Date: Apr. 29, 2004

(54) Methods of treating infection using anitbiotics and glycogen phosphorylase inhibitors

(75) Inventors: Judith L. Treadway, Mystic, CT (US); Joyce Anne Sutcliffe, Branford, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/623,032

(22) Filed: Jul. 16, 2003

Related U.S. Application Data
(60) Provisional application No. 60/396,960, filed on Jul. 18, 2002.

(51) Int. Cl. A61K 31/454; A61K 31/405

(52) U.S. Cl. 514/323; 514/414

(57) ABSTRACT

The invention provides methods of treating infection, such as Chlamydia pneumoniae infection, in a mammal comprising administering to a mammal effective amounts of azithromycin and a glycogen phosphorylase inhibitor. The invention also provides methods of treating atherosclerosis by administration of effective amounts of azithromycin and a glycogen phosphorylase inhibitor. Pharmaceutical compositions and kits are also provided.

### FIELD OF THE INVENTION

This invention relates to the use of the antibiotic azithromycin in combination with a glycogen phosphorylase inhibitor for the treatment of infections.

### BACKGROUND OF THE INVENTION

Glycogenolysis in tissues, whereby glycogen is cleaved to release gluclose-1 phosphate, is catalyzed by glycogen phosphorylase (GP). In humans, three isoforms of this enzyme have been identified: the liver isoform (HLGP), the muscle isoform (HMGP), and the brain isoform (HBGP). These isoforms are products of three separate genes and have 80-83% amino acid identity (C. B. Newgard, D. R. Littman, C. van Gendered, M. Smith and R. J. Fletterick, J. Biol. Chem. 263:3850-3857, 1988). Glycogen phosphorylase is also present in bacteria.

Glycogen phosphorylase inhibitors that have been reported to date include glucose and glucose analogs (e.g., Martin, J. L. et al., Biochemistry 1991, 30, 101 01), caffeine and other purine analogs (e.g., Kasvinsky, P. J. et al. J. Biol. Chem. 1978, 253, 3343-3351 and 9102-9106, and inhibitors of the type described by Oikonomakos, N. G. et al., Protein Sci 1999, 8, 1930-1945.

Glycogen phosphorylase inhibitors are useful in the treatment of diabetes mellitus. For example, International Patent publications WO 96139384 and WO 96/39385, both published Dec. 12, 1996, describe use of substituted N-(indole-2-carbonyl-) amides and derivatives for treatment of diabetes. These compounds are also described as useful treatment of atherosclerosis, hyperinsulinemia, hypercholesterolemia, hypertension, hyperlipidemia, and in prevention of myocardial ischemic injury.

U.S. Pat. No. 5,952,322 describes the use of glycogen phosphorylase inhibitors, such as those described in WO 96/39384 and WO 96/39385, to reduce tissue damage associated with non-cardiac ischemia.

U.S. Pat. No. 5,882,885, issued Mar. 16, 1999 refers to antagonists and agonists of streptococcal glycogen phosphorylase as useful in the treatment of otitis media, conjunctivitis, pnumonia, bacteremia, meningitis, sinusitis, pleural emphysema and endocarditis.

### SUMMARY OF THE INVENTION

The invention is directed to methods of treating infection in a mammal comprising administering to a mammal in need of such treatment effective amounts of the antibiotic azithromycin and a glycogen phosphorylase inhibitor. Preferably, the infection is a bacterial infection.

Another aspect of the invention provides methods of treating Chlamydia pneumoniae infection comprising administering to a mammal comprising administering to a mammal in need of such treatment effective amounts of azithromycin and a glycogen phosphorylase inhibitor.

A further aspect of the invention provides methods of treating atherosclerosis comprising administering to a mammal in need of such treatment effective amounts of azithromycin and a glycogen phosphorylase inhibitor or a pharmaceutically acceptable salt thereof or prodrug thereof.

Preferred glycogen phosphorylase inhibitors for use in the methods of the invention include 5-chloro-1H-indole-2-carboxylic acid [(1S)-(4-fluorobenzyl)-2-(4-hydroxypiperidin-1-yl)-2-oxoethyl]amide and 5-chloro-1H-indole-2-carboxylic acid [(1S)-benzyl-3-((BR,4S)-dihydroxypyrrolidin-1-yl)-(2R)-hydroxy-3-oxopropyl]amide.

Preferably, the azithromycin and glycogen phosphorylase inhibitor are administered in synergistic effective amounts.

The invention is also directed to pharmaceutical compositions comprising, in effective amounts, azithromycin and glycogen phosphorylase inhibitor, or a pharmaceutically acceptable salt thereof or prodrug thereof.

Yet another aspect of the invention is directed to kits comprising: a) azithromycin or a pharmaceutically acceptable salt thereof and a pharmaceutically acceptable carrier or diluent in a first unit dosage form; b) a glycogen phosphorylase inhibitor or a pharmaceutically acceptable salt thereof and a pharmaceutically acceptable carrier or diluent in a second unit dosage form; and c) a container.

A further aspect of the invention is directed to kits comprising azithromycin and instructions for administering a glycogen phosphorylase inhibitor to a mammal.

An additional aspect of the invention is directed to kits comprising a glycogen phosphorylase inhibitor and instructions for administering azithromycin to a mammal.

### DETAILED DESCRIPTION OF THE INVENTION

The invention provides methods of treating infection in a mammal comprising administering to a mammal having an infection effective amounts of the antiobiotic azithromycin and a glycogen phosphorylase inhibitor.

Azithromycin is the U.S.A.N. (generic name) for 9a-aza-9a-methyl-9-deoxo-9a-homoerythromycin A, a broad spectrum antimicrobial compound derived from erythromycin A. Azithromycin and its synthesis are described in Bright, U.S. Pat. No. 4,474,768 and Kobrehel et al., U.S. Pat. No. 4,517,359. These patents disclose that azithromycin and certain derivatives thereof possess antimicrobial properties and are accordingly useful as antibiotics. Azithromycin is in the azalide subclass of macrolide antibiotics. Azithromycin is distributed by Pfizer, Inc., New York, N.Y.

The invention contemplates the use of any compound that is a glycogen phosphorylase inhibitor. Glycogen phosphorylase inhibitors useful in the methods of the invention include the glycogen phosphorylase inhibitors of U.S. Pat. No. 6,107,329 issued Aug. 22, 2000 and U.S. Pat. No. 6,297,269 issued Oct. 2, 2001, the disclosures of each of which are hereby incorporated by reference. Preferably, the glycogen phosphorylase inhibitor is a compound of Formula I or Formula IA that is effective in treating or preventing infection. Compounds of Formula I and Formula IA have the following structures:

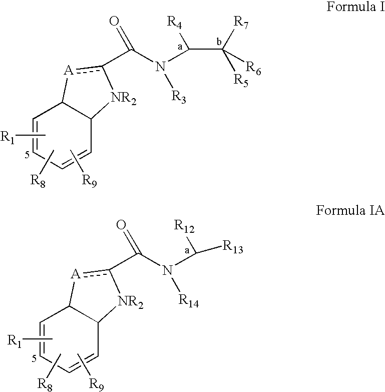

and the pharmaceutically acceptable salts and prodrugs thereof; wherein:

the dotted line (---) is an optional bond;

A is —C(H)═, —C((C₁-C₄)alkyl= or —C(halo)= when the dotted line (---) is a bond, or A is methylene or —CH((C₁-C₄)alkyl)- when the dotted line (---) is not a bond;

R₁, R₈, or R₉ are each independently H, halo, 4-, 6- or 7-nitro, cyano, (C₁-C₄)alkyl,

(C₁-C₄)alkoxy, fluoromethyl, difluoromethyl or trifluoromethyl;

R₂ is H;

R₃ is H or (C₁-C₅)alkyl;

R₄ is H, methyl, ethyl, n-propyl, hydroxy(C₁-C₃)alkyl, (C₁-C₃)alkoxy(C₁-C₃)alkyl, phenyl(C₁-C₄)alkyl, phenylhydroxy(C₁-C₄)alkyl, phenyl(C₁-C₄)alkoxy(C₁-C₄)alkyl, thien-2- or -3-yl(C₁-C₄)alkyl or fur-2- or -3-yl(C₁-C₄)alkyl wherein said R₄ rings are mono-, di- or tri-substituted independently on carbon with H, halo, (C₁-C₄)alkyl, (C₁-C₄)alkoxy, trifluoromethyl, hydroxy, amino or cyano; or

R₄ is pyrid-2-, -3- or -4-yl(C₁-C₄)alkyl, thiazol-2-, -4- or -5-yl(C₁-C₄)alkyl, imidazol -1-, -2-, -4- or -5-yl(C₁-C₄)alkyl, pyrrol-2- or -3-yl(C₁-C₄)alkyl, oxazol-2-, -4- or -5-yl-(C₁-C₄)alkyl pyrazol-3-, -4- or -5-yl(C₁-C₄)alkyl, isoxazol-3-, -4- or -5-yl(C₁-C₄)alkyl, isothiazol-3-, -4- or -5-yl(C₁-C₄)alkyl, pyridazin-3- or -4-yl-(C₁-C₄)alkyl, pyrimidin-2-, -4-, -5- or -6-yl(C₁-C₄)alkyl, pyrazin-2- or -3-yl(C₁-C₄)alkyl or 1,3,5-triazin-2-yl(C₁-C₄)alkyl, wherein said preceding R₄ heterocycles are optionally mono- or di-substituted independently with halo, trifluoromethyl, (C₁-C₄)alkyl, (C₁-C₄)alkoxy, amino or hydroxy and said mono-or di-substituents are bonded to carbon;

R₅ is H, hydroxy, fluoro, (C₁-C₅)alkyl, (C₁-C₅)alkoxy, (C₁-C₆)alkanoyl,

amino(C₁-C₄)alkoxy, mono-N- or di-N,N-(C₁-C₄)alkylamino(C₁-C₄)alkoxy,

carboxy(C₁-C₄)alkoxy, (C₁-C₅)alkoxycarbonyl(C₁-C₄)alkoxy, benzyloxycarbonyl(C₁-C₄)alkoxy, or carbonyloxy wherein said carbonyloxy is carbon-carbon linkined with phenyl, thiazolyl, imidazolyl, 1H-indolyl, furyl, pyrrolyl, oxazolyl, pyrazolyl, isoxazolyl, isothiazolyl, pyridazinyl, pyrimidinyl, pyrazinyl or 1,3,5-triazinyl and wherein said preceding R₅ rings are optionally mono-substituted with halo, (C₁-C₄)alkyl, (C₁-C₄) alkoxy, hydroxy, amino or trifluoromethyl and said mono-substituents are bonded to carbon;

R₇ is H, fluoro or (C₁-C₆)alkyl; or

R₅ and R₇ can be taken together to be oxo;

R₆ is C(O)R_{10;}

R₁₀ is piperazin-1-yl, 4-(C₁-C₄)alkylpiperazin-1-yl, 4-formylpiperazin-1-yl, morpholino, thiomorpholino, 1-oxothiomorpholino, 1,1-dioxo-thiomorpholino, thiazolidin-3-yl, 1-oxo-thiazolidin-3-yl, 1,1-dioxo-thiazolidin-3-yl, 2-(C₁-C₆)alkoxycarbonylpyrrolidin-1-yl, oxazolidin-3-yl or 2(R)-hydroxymethylpyrrolidin-1-yl; or

R₁₀ is 3- and/or 4-mono-or di-substituted oxazetidin-2-yl, 2-, 4-, and/or 5-mono- or di-substituted oxazolidin-3-yl, 2-, 4- and/or 5-mono- or di-substituted thiazolidin-3-yl, 2-, 4- and/or 5-mono- or di-substituted 1-oxothiazolidin-3-yl, 2-, 4-, and/or 5-mono- or di-substituted 1,1-dioxothiazolidin-3-yl, 3- and/or 4-, mono- or di-substituted pyrrolidin-1-yl, 3,4-and/or 5-, mono-, di- or tri-substituted piperidin-1-yl, 3-, 4-, and/or 5-mono-, di-, or tri-substituted piperazin-1-yl, 3-substituted azetidin-1-yl, 4 and/or 5-, mono- or di-substituted, 1,2-oxazinan-2-yl, 3-and/or 4-mono- or di-substituted pyrazolidin-1-yl, 4- and/or 5-, mono- or di-substituted isoxazolidin-2-yl, 4 and/or 5-, mono- and/or di-substituted isothiazolidin-2 yl wherein said R₁₀ substituents are independently H, halo (C₁-C₅)-alkyl, hydroxy, amino, mono-N- or di-N,N-(C₁-C₅)alkylamino, formyl, oxo, hydroxyimino, (C₁-C₅)alkoxy, carboxy, carbamoyl, mono-N-or di-N,N-(C₁-C₄)alkylcarbamoyl, (C₁-C₄)alkoxyimino, (C₁-C₄)alkoxy-methoxy, (C₁C₆)alkoxy-carbonyl, carboxy(C₁-C₅)alkyl or hydroxy(C₁-C₅)alkyl;

R₁₂ is H, methyl, ethyl, n-propyl, hydroxy(C₁-C₃)alkyl, (C₁-C₃)alkoxy(C₁C₃)alkyl, phenyl(C₁-C₄)-alkyl, phenylhydroxy(C₁-C₄)alkyl, (phenyl)((C₁-C₄)-alkoxy)(C₁C₄)alkyl, thien-2- or -3-yl(C₁-C₄)alkyl or fur-2-or -3-yl(C₁-C₄)alkyl wherein said R₁₂ rings are mono-, di- or tri-substituted independently on carbon with H, halo, (C₁C₄)alkyl, (C₁-C₄)alkoxy, trifluoromethyl, hydroxy, amino, cyano or 4,5-dihydro-1H-imidazol-2-yl; or

R₁₂ is pyrid-2-, -3- or -4-yl(C₁-C₄)alkyl, thiazol-2-, 4- or -5-yl(C₁-C₄)alkyl, imidazol-2-,

-4- or -5-yl(C₁-C₄)alkyl, pyrrol-2- or -3-yl(C₁-C₄)alkyl, oxazol-2-, 4- or -5-yl(C₁C₄)alkyl, pyrazol-3-, 4- or -5-yl(C₁-C₄)alkyl, isoxazol-3-, -4- or -5-yl(C₁-C₄)alkyl, isothiazol-3-, 4- or -5-yl(C₁-C₄)alkyl, pyridazin-3- or -4-yl(C₁-C₄)alkyl, pyrimidin-2-, 4-, 5- or -6-yl(C₁-C₄)— alkyl, pyrazin-2-or -3-yl(C₁-C₄)alkyl, 1,3,5-trizin-2-yl(C₁-C₄)alkyl or indol-2-(C₁-C₄)alkyl, wherein said preceding R₁₂ heterocycles are optionally mono- or di-substituted independently with halo, trifluoromethyl, (C₁-C₄)alkyl, (C₁-C₄)alkoxy, amino, hydroxy or cyano and said substituents are bonded to carbon; or

R₁₂ is R₁₁-carbonyloxymethyl, wherein said R₁₁ is phenyl, thiazolyl, imidazolyl, 1H-indolyl, furyl, pyrrolyl, oxazolyl, pyrazolyl, isoxazolyl, isothiazolyl, pyridyl, pyridazinyl, pyrimidinyl, pyrazinyl or 1,3,5-triazinyl and wherein said preceding R₁₁ rings are optionally mono- or di-substituted independently with halo, amino, hydroxy, (C₁-C₄)alkyl, (C₁-C₄)alkoxy or trifluoromethyl and said mono- or di-substituents are bonded to carbon;

R₁₃ is H, methyl, ethyl, n-propyl, hydroxymethyl, or hydroxyethyl;

R₁₄ is C(O)R₁₅;

R₁₅ is morpholino, thiomorpholino, 1-oxothiomorpholino, 1,1-dioxothiomorpholino, thiazolidin-3-yl, 1-oxothiazolidin-3-yl, 1,1-dioxothiazolidin-3-yl, pyrrolidin-1-yl, piperidin-1-yl, piperazin-1-yl, piperazin-4-yl; azetidin-1-yl, 1,2-oxazinan-2-yl, pyrazolidin-1-yl, isoxazolidin-2-yl, isothiazolidin-2-yl, 1,2,-oxazetidin-2-yl; oxazolidin-3-yl, 3,4-dihydroisoquinolin-2-yl, 1,3-dihydroisoindol-2-yl, 3,4-dihydro-2H-quinol-1-yl, 2,3-dihydro-benzo[1,4]oxazin4-yl, 2,3-dihydro-benzo[1,4]-thiazine-4-yl, 3,4-dihydro-2H-quinoxalin-1-yl, 3,4-dihydro-benzo[c][1,2]oxazin-1-yl, 1,4-dihydro-benzo[d][1,2]oxazin-3-yl, 3,4-dihydro-benzo[e][1,2]-oxazin-2-yl, 3H-benzo[d]isoxazol-2-yl, 3H-benzo[c]isoxazol-1-yl or azepan-1-yl, wherein said R₁₅ rings are optionally mono-, di- or tri-substituted independently with halo,

(C₁-C₅)alkyl, (C₁-C₅)alkoxy, hydroxy, amino, mono-N- or di-N,N-(C₁-C₅)alkylamino, formyl, carboxy, carbamoyl, mono-N- or di-N,N-(C₁-C₅)alkylcarbamoyl, (C₁-C₆)alkoxy(C₁-C₃)alkoxy, (C₁-C₅)alkoxycarbonyl, benzyloxycarbonyl, (C₁C₅)alkoxycarbonyl(C₁-C₅)alkyl, (C₁-C₄)alkoxy-carbonylamino, carboxy(C₁-C₅)alkyl, carbamoyl(C₁-C₅)alkyl, mono-N- or di-N,N-(C₁-C₅)alkyl-carbamoyl(C₁-C₅)alkyl, hydroxy(C₁-C₅)alkyl, (C₁-C₄)alkoxy(C₁-C₄)alkyl, amino(C₁-C₄)alkyl, mono-N- or di-N,N-(C₁-C₄)alkylamino(C₁-C₄)alkyl, oxo, hydroxyimino or (C₁-C₆)alkoxyimino and wherein no more than two substituents are selected from oxo, hydroxyimino or (C₁-C₆)alkoxyimino and oxo, hydroxyimino or (C₁-C₆)alkoxyimino are on nonaromatic carbon; and wherein said R₁₅ rings are optionally additionally mono- or di-substituted independently with (C₁-C₅)alkyl or halo.

A group of preferred compounds of Formula I consists of those compounds wherein:

R₁ is 5-H, 5-halo, 5-methyl or 5-cyano;

R₈ and R₉ are each independently H or halo;

A is —C(H)═;

R₂ and R₃ are H;

R₄ is phenyl(C₁-C₂) alkyl wherein said phenyl groups are mono-, di- or tri-substituted independently with H or halo or mono- or di-substituted independently with H, halo (C₁-C₄)alkyl, (C₁-C₄)alkoxy, trifluoromethyl, hydroxy, amino or cyano; or

R₄ is thien-2- or -3-yl(C₁-C₂)alkyl, pyrid-2-, -3- or -4-yl(C₁-C₂)alkyl, thiazol-2-,4- or -5yl(C₁-C₂)alkyl, imidazol -1-, -2-, 4- or -5-yl(C₁-C₂)alkyl, fur-2- or -3-yl(C₁C₂)alkyl, pyrrol-2- or -3-yl(C₁-C₂)alkyl, oxazol-2-, 4- or -5-yl-(C₁-C₂)alkyl, pyrazol-3-, -4- or -5-yl(C₁-C₂)alkyl, isoxazol-3-, 4- or -5-yl(C₁-C₂)alkyl wherein said preceding R₄ heterocycles are optionally mono- or di-substituted independently with halo, trifluoromehtyl, (C₁-C₄)alkyl, (C₁-C₄)alkoxy, amino or hydroxy and said mono- or di-substituents are bonded to carbon;

R₅ is hydroxy; and

R₇ is H.

Within the above group of preferred compounds of Formula I is a second group of especially preferred compounds wherein

the carbon atom labeled a has (S) stereochemistry;

the carbon atom labeled b has (R) stereochemistry;

R₄ is phenyl(C₁-C₂) alkyl, thien-2-yl(C₁-C₂)alkyl, thien-3-yl-(C₁-C₂)alkyl, fur-2-yl-(C₁-C₂)alky- I or fur-3-yl-(C₁-C₂)alkyl wherein said rings are mono- or di-substituted independently with H or fluoro; and

R₁₀ is morpholino, 4-(C₁-C₄) alkylpiperazin-1-yl, 3-substituted azetidin-1-yl, 3- and/or 4-mono- or di-substituted pyrrolidin-1-yl, 4- and/or 5-mono- or di-substituted isoxazolidin-2-yl, 4- and/or 5-, mono- or di-substituted 1,2-oxazinan-2-yl wherein said substituents are each independently H, halo, hydroxy, amino, mono-N-or di-N,N-(C₁-C₆)alkylamino, oxo, hydroxyimino or alkoxy.

Within the above group of especially preferred compounds are the particularly preferred compounds:

5-Chloro-1H-indole-2-carboxylic acid [(1S-benzyl-(2R)-hydroxy-3-(4- -methylpiperazin-1-yl)-3-oxo-propyl]-amide hydrochloride,

5-Chloro-1H-indole-2-carboxylic acid [(1S)-benzyl-(2R)-hydroxy-3-(3-hydroxyazetidin-1-yl)-3-oxo-propyl]-amide,

5-Chloro-1H-indole-2-carboxylic acid ((1S)-benzyl-(2R)-hydroxy-3-isoxazolidin-2-yl-3-oxo-propyl)-amide,

5-Chloro-1H-indole-2-carboxylic acid ((1S)-benzyl-(2R)-hydroxy-3-[1-,2]oxazinan-2-yl-3-oxo-propyl)-amide,

5-Chloro-1H-indole-2-carboxylic acid [(1S)-benzyl-(2R)-hydroxy-3-((3S)hydroxypyrrolidin-1-yl)-3-oxo-propyl]-amide,

5-Chloro-1H-indole-2-carboxylic acid [(1S)-benzyl-3-((3S,4S)-dihydroxypyrrolidin-1-yl)-(2R)-hydroxy-3-oxo-propyl]-amide,

5-Chloro-1H-indole-2-carboxylic acid [(1S)-benzyl-3-(cis-3,4-dihydroxypyrrolidin-1-yl)-(2R)-hydroxy-3-oxo-propyl]-amide;

5-Chloro-1H-indole-2-carboxylic acid ((1S)-benzyl-(2R)-hydroxy-3-morpholin-4-yl-3-oxo-propyl)-amide; and

5-Chloro-1H-indole-2-carboxylic acid [(1S)-benzyl-3-((3R,4S)dihydroxy-pyrrolidin-1-yl)-(2R)-hydroxy-3-oxopropyl]amide.

Within the above group of especially preferred compounds of Formula I are compounds wherein:

a. R₁ is 5-chloro; R₈ and R₉ are H; R₄ is benzyl; and R₁₀ is 4-methylpiperazin-1-yl;

b. R₁ is 5-chloro; R₈ and R₉ are H; R₄ is benzyl; and R₁₀ is 3-hydroxyazetidin-1-yl;

c. R₁ is 5-chloro; R₈ and R₉ are H; R₄ is benzyl; and R₁₀ is isoxazolidin-2-yl;

d. R₁ is 5-chloro; R₈ and R₉ are H; R₄ is benzyl; and R₁₀ is (1,2)-oxazinan-2-yl;

e. R₁ is 5-chloro; R₈ and R₉ are H; R₄ is benzyl; and R₁₀ is 3(S)-hydroxypyrrolidin-1-yl;

f. R₁ is 5-chloro; R₈ and R₉ are H; R₄ is benzyl; and R₁₀ is (3S,4S)-dihydroxypyrrolidin-1-yl;

g. R₁ is 5-chloro; R₈ and R₉ are H; R₄ is benzyl; and R₁₀ is cis-3,4-dihydroxypyrrolidin-1-yl;

h. R₁ is 5-chloro; R₈ and R₉ are H; R₄ is benzyl; and R₁₀ is morpholino; and

i. R₁ is 5-chloro; R₈ and R₉ are H; R₄ is benzyl; and R₁₀ is (3R,4S)-dihydroxypyrrolidin-1-yl

Another group of preferred compounds of Formula I are those wherein

R₁ is H, halo, methyl or cyano;

R₈ and R₉ are each independently H or halo;

A is —C(H)═;

R₂ and R₃ are H;

R₄ is phenyl (C₁-C₂)alkyl wherein said phenyl groups are mono-, di- or tri-substituted independently with H or halo or mono- or di-substituted independently with H, halo, (C₁-C₄)alkyl, (C₁-C₄)alkoxy, trifluoromethyl, hydroxy, amino or cyano; or R₄ is thien-2- or -3-yl(C₁-C₂)alkyl, pyrid-2-, -3- or 4-yl(C₁-C₂)alkyl, thiazol-2-, -4- or -5-yl(C₁-C₂)alk-yl, imidazol -1-, -2-, -4- or -5-yl(C₁-C₂)alkyl, fur-2- or -3-yl(C₁-C₂)alkyl, pyrrol-2- or -3-yl(C₁-C₂)alkyl, oxazol-2-, 4- or -5-yl(C₁-C₂)alkyl, pyrazol-3-, -4- or -5-yl(C₁-C₂)alkyl, isoxazol-3-, 4- or -5-yl(C₁-C₂) alk-yl wherein said preceding R₄ heterocycles are optionally mono-or-di-substituted

independently with halo, trifluoromethyl, (C₁-C₄)alkyl, (C₁-C₄)alkoxy, amino or hydroxy and said mono- or di-substituents are bonded to carbon;

R₅ is fluoro, (C₁-C₄)alkyl, (C₁-C₅)alkoxy, amino(C₁-C₄)alkoxy, mono-N- or di-N,N-(C₁-C₄)alkylamino(C₁-C₄)alkoxy, carboxy(C₁-C₄)alkoxy, (C₁-C₅)alkoxycarbonyl(C₁-C₄)alkoxy, benzyloxycarbonyl(C₁-C₄)alkoxy; and

R₇ is H, fluoro or (C₁-C₆)alkyl.

A group of preferred compounds of Formula 1 A consists of those compounds wherein

R₁ is 5-H, 5-halo, 5-methyl, 5-cyano or 5-trifluoromethyl;

R₈ and R₉ are each independently H or halo;

A is —C(H)═;

R₂ and R₃ are H;

R₁₂ is H, methyl, phenyl (C₁-C₂)alkyl, wherein said phenyl groups are mono- or di-substituted independently with H, halo (C₁-C₄)alkyl, (C₁-C₄)alkoxy, trifluoromethyl, hydroxy, amino or cyano and wherein said R₁₂ groups are optionally additionally mono-substituted with halo; or

R₁₂ is thien-2- or -3-yl(C₁-C₂)alkyl, pyrid-2-, -3- or -4-yl(C₁-C₂)alkyl, thiazol-2-, -4- or -5-yl(C₁-C₂)alkyl, imidazol-2-, -4- or -5-yl(C₁-C₂)alkyl, fur-2- or -3-yl(C₁-C₂)alkyl,

pyrrol-2- or -3-yl(C₁-C₂)alkyl, oxazol-2-, -4- or -5-yl(C₁-C₂)alkyl, pyrazol-3-, -4- or -5-yl(C₁-C₂)alkyl, isoxazol-3-, -4- or -5-yl(C₁-C₂)alkyl, isothiazol-3-, -4- or -5-yl(C₁-C₂)alkyl, pyridazin-3- or -4-yl(C₁-C₂)alkyl, pyrimidin-2-, -4-, -5- or -6-yl(C₁-C₂)alkyl, pyrazin-2- or -3-yl(C₁-C₂)alkyl or 1,3,5-triazin-2-yl(C₁-C₂)alkyl wherein said preceding R₁₂ heterocycles are optionally mono- or di-substituted independently with halo, trifluoromehtyl, (C₁-C₄)alkyl, (C₁-C₄)alkoxy, amino or hydroxy and said mono- or di-subtituents are bonded to carbon; and

R₁₃ is H.

Within the above group of preferred compounds of Formula IA is a group of especially preferred compounds wherein:

R₁₂ is H, phenyl(C₁-C₂)alkyl, thien-2- or -3-yl(C₁-C₂)alkyl, fur-2- or -3-yl(C₁C₂)alkyl wherein said R₁₂ rings are mono- or di-substituted independently with H or fluoro; and

R₁₅ is morpholino, thiomorpholino, 1-oxothiomorpholino, 1, 1-dioxothiomorpholino, thiazolidin-3-yl, 1-oxothiazolidin-3-yl, 1,1-dioxothiazolidin-3-yl, pyrrolidin-1-yl, piperidin-1-yl, piperazin-1-yl, piperazin-4-yl, azetidin-1-yl, 1,2-oxazinan-2-yl, isoxazolidin-2-yl, isothiazolidin-2-yl, 1,2-oxazetidin-2-yl, oxazolidin-3-yl, 1,3-dihydroisoindol-2-yl, or azepan-1-yl, wherein said R₁₅ rings are optionally mono- or di-substituted independently with halo, (C₁-C₅)alkyl, (C₁-C₅)alkoxy, hydroxy, amino, mono-N-or di-N- ,N-(C₁-C₅)alkylamino, formyl, carboxy, carbamoyl, mono-N- or di-N,N-(C₁-C₅)alkylcarbamoyl, (C₁-C₅)alkoxycarbonyl, hydroxy(C₁-C₅)alkyl, amino(C₁-C₄)alkyl, mono-N-or di-N,N-(C₁-C₄)alkylamino(C₁-C₄)alkyl, oxo, hydroxyimino or (C₁-C₆)alkoxyimino with the proviso that only the R₁₅ heterocycles thiazolidin-3-yl, pyrrolidin-1-yl, piperidin-1-yl, piperazin-1-yl, piperazin-4-yl, azetidin-1-yl, 1,2-oxazinan-2-yl, isoxazolidin-2-yl, or oxazolidin-3-yl are optionally mono- or di-substituted with oxo, hydroxyimino, or (C₁-C₆)alkoxyimino; and wherein said R₁₅ rings are optionally additionally mono- or di-substituted independently with (C₁-C₅)alkyl.

Within the above group of especially preferred compounds are the compounds:

5-Chloro-1H-indole-2-carboxylic acid [(1S)-benzyl-2-(3-hydroxyiminopyrrolidin-1-yl)-2-oxo-ethyl]-amide,

5-Chloro-1H-indole-2-carboxylic acid [2-(cis-3,4-dihydroxypyrrolidin-1-yl)-2-oxo-ethyl]-amide,

5-Chloro-1H-indole-2-carboxylic acid [2-((3S,4S)-dihydroxypyrrolidin-1-yl)-2-oxo-ethyl]-amide,

5-Chloro-1H-indole-2-carboxylic acid [(1S)-benzyl-2-(cis-3,4-dihydroxypyrrolidin-1-yl)-2-oxo-ethyl]-amide,

5-Chloro-1H-indole-2-carboxylic acid [2-(1,1-dioxothiazolidin-3-yl)-2-oxo-ethyl]-amide,

5-Chloro-1H-indole-2-carboxylic acid (2-oxo-2-thiazolidin-3-yl-ethyl-)amide,

5-Chloro-1H-indole-2-carboxylic acid [(1S)-(4-flurobenzyl)-2-(4-hydroxypiperidin-1-yl)-2-oxo-ethyl]-amide,

5-Chloro-1H-indole-2-carboxylic acid [(1S)-benzyl-2-((3RS)-hydroxypiperidin-1-yl)-2-oxo-ethyl]-amide,

5-Chloro-1H-indole-2-carboxylic acid [2-oxo-2-((1RS)-oxo-1-thiazolidin-3-yl)ethyl]-amide,

5-Chloro-1H-indole-2-carboxylic acid [(1S)-(2-fluoro-benzyl)-2-(4-hydroxypiperidin-1-yl)-2-oxo-ethyl]-amide,

5-Chloro-1H-indole-2-carboxylic acid [(1S)-benzyl-2-((3S,4S)-dihydroxypyrrolidin-1-yl)-2-oxo-ethyl]-amide,

5-Chloro-1H-indole-2-carboxylic acid [(1S)-benzyl-2-(3-hydroxy-azetidin-1-yl)-2-oxoethyl]-amide,

5-Chloro-1H-indole-2-carboxylic acid [(1S)-benzyl-2-(3-hydroxyiminoazetidin-1-yl)-2-oxo-ethyl]-amide,

5-Chloro-1H-indole-2-carboxylic acid [(1S)-benzyl-2-(4-hydroxyimino-piperidin-1-yl)-2-oxo-ethyl]-amide, and

5-Chloro-1H-indole-2-carboxylic acid [1-benzyl-2-(3-hydroxypyrrolidin-1-yl)-2-oxo-ethyl]amide.

Within the group of especially preferred compounds of Formula IA is a group of particularly preferred compounds wherein:

R₁₂ is H; and

R₁₅ is thiazolidin-3-yl, 1-oxo-thiazolidin-3-yl, 1,1-dioxo-thiazolidin-3-yl or oxazolidin-3-yl or said R₁₅ substituents optionally mono- or di-substituted independently with carboxy, (C₁-C₅)alkoxycarbonyl, hydroxy(C₁-C₃)alkyl, amino(C₁-C₃)alkyl, mono-N- or di-N,N-(C₁-C₃)alkylamino (C₁-C₃) alkyl or

R₁₅ is mono-or di-substituted pyrrolidin-1-yl wherein said substituents are independently carboxy, (C₁-C₅)alkoxycarbonyl, (C₁-C₅)alkoxy, hydroxy, hydroxy(C₁C₃)alkyl, amino, amino(C₁-C₃)alkyl, mono-N- or di-N,N-(C₁-C₃)alkylamino-(C₁-C₃)alkyl or mono-N- or di-N,N-(C₁-C₄)alkylamino; and

the R₁₅ rings are optionally additionally independently disubstituted with (C₁-C₅)alkyl.

Preferred compounds with the immediately preceding group of compounds are those wherein:

a. R₁ is 5-chloro; R₈ and R₉ are H; and R₁₅ is cis-3,4-dihydroxy-pyrrolidin-1-yl;

b. R₁ is 5-chloro; R₈ and R₉ are H; and R₁₅ is (3S,4S)-dihydroxy-pyrrolidin-1-yl;

c. R₁ is 5-chloro; R₈ and R₉ are H; and R₁₅ is 1,1-dioxo-thiazolidin-3-yl;

d. R₁ is 5-chloro; R₈ and R₉ are H; and R₁₅ is thiazolidin-3-yl; and

e. R₁ is 5-chloro; R₈ and R₉ are H; and R₁₅ is 1-oxo-thiazolidin-3-yl.

Within the above group of especially preferred compounds of Formula IA is another group of particularly preferred compounds wherein:

R₁₅ is phenylmethyl, thien-2- or -3-ylmethyl wherein said R₁₅ rings are optionally mono- or di-substituted with fluoro; and

R₁₅ is thiazolidin-3-yl, 1-oxo-thiazolidin-3-yl, 1,1-dioxo-thiazolidin-3-yl or oxazolidin-3-yl or said R₁₅ substituents optionally mono- or di-substituted independently with carboxy or (C₁-C₅)alkoxycarbonyl, hydroxy(C₁-C₃)alkyl-, amino(C₁C₃)alkyl or mono-N-or di-N,N-(C₁-C₃)alkylamino (C₁-C₃)alkyl, or R₁₅ is mono-or di-substituted azetidin-1-yl or mono- or di-substituted pyrrolidin-1-yl or mono- or di-substituted piperidin-1-yl wherein said substituents are independently carboxy, (C₁-C₅)alkoxycarbonyl, hydroxy(C₁-C₃)alkyl, amino(C₁C₃)alkyl, mono-N- or di-N,N-(C₁-C₃)alkylamino(C₁-C₃)alkyl, hydroxy, (C₁-C₅)alkoxy, amino, mono-N- or di-N,N-(C₁-C₅)alkylamino, oxo, hydroxyimino or (C₁-C₅)alkoxyimino; and

the R₁₅ rings are optionally additionally mono- or di-substituted independently with (C₁-C₅)alkyl.

Preferred compounds within the immediately preceding group of particularly preferred compounds of Formula IA are compounds wherein

a. R₁ is 5-chloro; R₈ and R₉ are H; R₁₂ is 4-fluorobenzyl; R₁₅ is 4-hydroxypiperidin-1-yl; and the stereochemistry of carbon(a) is (S);

b. R₁ is 5-chloro; R₈ and R₉ are H, R₁₂ is benzyl; R₁₅ is 3-hydroxypiperidin-1-yl; and

the stereochemistry of carbon (a) is (S);

c. R₁ is 5-chloro; R₈ and R₉ are H; R₁₂ is benzyl; R₁₅ is cis-3,4-dihydroxypyrrolidin-1-yl; and the stereochemistry of carbon (a) is S;

d. R₁ is 5-chloro; R₈ and R₉ are H; R₁₂ is benzyl; R₁₅ is 3-hydroxyiminopyrrolidin-1-yl; and the stereochemistry of carbon (a) is (S);

e. R₁ is 5-chloro; R₈ and R₉ are H; R₁₂ is 2-fluorobenzyl; R₁₅ is 4-hydroxypiperidin-1-yl; and the stereochemistry of carbon (a) is (S);

f. R, is 5-chloro; R₈ and R₉ are H; R₁₂ is benzyl; R₁₅ is (3S,4S)-dihydroxypyrrolidin-1-yl; and the stereochemistry of carbon (a) is (S);

g. R₁ is 5-chloro; R₈ and R₉ are H; R₁₂ is benzyl; R₁₅ is 3-hydroxyazedidin-1-yl; and

the stereochemistry of carbon (a) is (S);

h. R₁ is 5-chloro; R₈ and R₉ are H; R₁₂ is benzyl; R₁₅ is 3-hydroxyiminoazetidin-1-yl; and the stereochemistry of carbon (a) is (S); and

i. R₁ is 5-chloro; R₈ and R₉ are H; R₁₂ is benzyl; R₁₅ is 4-hydroxyiminopiperidin-1-yl; and the stereochemistry of carbon (a) is (S).

The compounds of Formula I and IA can be prepared according to the methods of U.S. Pat. Nos. 6,107,329 and 6,297,269.

More preferably, the glycogen phosphorylase inhibitor is 5-chloro-1H-indole-2-carboxylic acid [(1S)-(4-fluorobenzyl)-2-(4-hydroxypiperidin-1-yl)-2-oxoethyl]amide or

5-chloro-1H-indole-2-carboxylic acid [(1S)-benzyl-3-((3R,4S)-dihydroxypyrrolidin-1-yl)-(2R)-hydroxy-3-oxopropyl]amide.

5-chloro-1H-indole-2-carboxylic acid [(1S)-(4-fluorobenzyl)-2-(4-hydroxypiperidin-1-yl)-2-oxoethyl]amide can be prepared according to the method found in U.S. Pat. No. 6,297,269.

5-chloro-1H-indole-2-carboxylic acid [(1S)-benzyl-3-((3R,4S)-dihydroxypyrrolidin-1-yl)-(2R)-hydroxy-3-oxopropyl]amide can be prepared according to the method found in U.S. Pat. No. 6,107,329.

The methods and pharmaceutical compositions of the invention are useful for treating mammals, including humans, farm animals such as cows, pigs and horses, and companion animals such as dogs and cats.

The methods of the invention can be used to treat bacterial, fungal, parasitic or viral infections.

The method of the invention is employed to treat bacterial infections and protozoa infections and disorders related to such infections that include the following: pneumonia, otitis media, sinusitis, bronchitis, tonsillitis, and mastoiditis related to infection by Streptococcus pneumoniae, Haemophilus influenzae, Moraxella catarrhalis, Staphylococcus aureus, or Peptostreptcoccus spp.; pharynigitis, rheumatic fever, and glomenulonephritis related to infection by Streptococcus pyogenes, Groups C and G streptococci, Clostridium diptheriae, or Actinobacillus haemolyticum; respiratory tract infections related to infection by Mycoplasma pneumoniae, Legionella pneumophila, Streptococcus pneumoniae, Haemophilus influenzae, or Chlamydia pneumoniae; uncomplicated skin and soft tissue infections, abcesses and osteomyelitis, and puerperal fever related to infection by Staphylococcus aureus, coagulase-positive staphylococci (i.e., S. epidermidis, S. hemolyticus, etc.), Streptococcus pyogenes, Streptococcus agalactiae, Streptococcal groups C-F (minute-colony streptococci), viridans streptococci, Corynebacterium minutissimum, Clostridium spp., or Bartonella henselae; uncomplicated acute urinary tract infections related to infection by Staphylococcus saprophyticus or Enterococcus spp.; urethritis and cervicitis; and sexually transmitted diseases related to infection by Chlamydia trachomatis, Haemophilus ducreyi, Treponema pallidum, Ureaplasma urealyticum, or Neiserria gonofrheae; toxin diseases related to infection by S. aureus (food poisoning and Toxic shock syndrome), or Groups A, B, and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by Chlamydia trachomatis, Neisseria gonorrhoeae, S. aureus, S. pneumoniae, S. pyogenes, or H. influenzae; disseminated Mycobacterium avium complex (MAC) disease related to infection by Mycobacterium avium, or Mycobacterium intracellulare; gastroenteritis related to infection by Campylobacter jejunr; intestinal protozoa related to infection by Cryptosporidium spp.; odontogenic infection related to infection by viridans streptococci; persistent cough related to infection by Bordetella pertussis; gas gangrene related to infection by Clostridium perfringens or Bacteroides spp.; atherosclerosis related to infection by Helicobacter pylori; Chlamydia pneumoniae, or Mycoplasma pneumoniae, dysentery related to infection by Shigella dysenteriae, and symptoms of infection by enterotoxigenic E. coli or Mycobacterium tuberculosis.

Bacterial infections and protozoa infections and disorders related to such infections that may be treated or prevented in animals include the following: bovine respiratory disease related to infection by Pasteurella haemolyticus, P. multocida, Mycoplasma bovis, or Bordetella spp.; cow enteric disease related to infection by E. coli or protozoa (i.e., coccidia, cryptosporidia, etc.); dairy cow mastitis related to infection by Staph. aureus, Strep. uberis, Strep. agalactiae, Strep. dysgalactiae, Klebsiella spp., Corynebacterium, or Enterococcus spp.; swine respiratory disease related to infection by Actinobacillus pleuropneumoniae, P. multocida, or Mycoplasma spp.; swine enteric disease related to infection by E coli, Lawsonia intracellularis, Salmonella, or Serpulina hyodysenteriae; cow footrot related to infection by Fusobacterium spp.; cow metritis related to infection by E. coli, cow hairy warts related to infection by Fusobacterium necrophorum or Bacteroides nodosus; cow pink-eye related to infection by Moraxella bovis; cow premature abortion related to infection by protozoa (i.e. neosporium); urinary tract infection in dogs and cats related to infection by E. coli; skin and soft tissue infections in dogs and cats related to infection by Staph. epidermidis, Staph intermedius, coagulase neg. Staph. or P. multocida; and dental or mouth infections in dogs and cats related to infection by Alcaligenes spp., Bacteroides spp., Clostridium spp., Enterobacter spp., Eubacteriu, Peptostreptococcus, Porphyromonas, or Prevotella.

The invention also encompasses treatment of bacteremia, meningitis, pleural empyema, malaria, river blindness, toxoplasmosis, and endocarditis. Other bacterial infections and protozoa infections and disorders related to such infections that may be treated or prevented in accord with the method of the present invention are referred to in J. P. Sanford et al., “The Sanford Guide To Antimicrobial Therapy,” 26^{th} Edition, (Antimicrobial Therapy, Inc., 1996).

In a preferred aspect, the methods of the invention are used to treat bacterial infection and disorders related to such infection, more preferably infection by Chlamydia spp and related disorders, most preferably infection by Chlamydia pneumonia and related disorders.

In another preferred aspect, the invention provides a method of treating atherosclerosis, especially atherosclerosis related to infection by Helicobacter pylori, Chlamydia pneumoniae, or Mycoplasma pneumonia, comprising administering effective amounts of azithromycin and a glycogen phosphorylase inhibitor to a mammal in need of such treatment.

In studies with the bacterial pathogen Chlamydia pneumonia, azithromycin in combination with the glycogen phosphorylase inhibitor 5-chloro-1H-indole-2-carboxylic acid [(1S)-(4-flurobenzyl)-2-(4-hydroxypiperidin-1-yl)-2-oxoethyl]amide or 5-chloro-1H-indole-2-carboxylic acid [(1S)-benzyl-3-((3R,4S)-dihydroxypyrrolidin-1-yl)-(2R)-hydroxy-3-oxopropyl]amide was found to have synergistic antibacterial effects. The synergistic antibacterial effects of glycogen phosphorylase inhibitors and azithromycin were observed for both minimum inhibitory concentration (MIC) and minimum bacteriocidal concentration (MBC). 5-chloro-1H-indole-2-carboxylic acid [(1S)-(4-fluorobenzyl)-2-(4-hydroxypiperidin-1-yl)-2-oxoetheyl]amide reduced the MIC and MBC for azithromycin by 3-10-fold. 5-chloro-1H-indole-2carboxylic acid [(1S)-benzyl-3-((3R,4S)-dihydroxy-pyrrolidin-1-yl)-(2R)-hydroxy-3-oxopropyl]amide reduced the MIC for azithromycin by 3-10-fold, and the MBC by 3-fold. The glycogen phosphorylase inhibitor's synergistic antibacterial effects with azithromycin were concentration-dependent, and unrelated to any non-specific effects on the host cell.

Administration of azithromycin with a glycogen phosphorlyase inhibitor could allow more favorable treatment options, reduced dose or frequency of the antibiotic or glycogen phosphorylase inhibitor administered, and the ability to treat patients at a more desirable therapeutic index, or with increased bacteriostatic efficacy (e.g., more likely to achieve effective MBC concentrations).

Preferably, in the practice of the methods of the invention, azithromycin and a glycogen phosphorylase inhibitor are administered to the mammal in synergistic effective amounts.

Where used herein, synergistic effective amounts of azithromycin and a glycogen phosphorylase inhibitor are amounts which, when administered to a mammal having, or suspected of having, an infection or related disease such as atherosclerosis, are sufficient to exhibit a greater action against the infection or related disease than the sum of the action that would be observed upon independent administration of the antibiotic and glycogen phosphorylase inhibitor alone.

In a further embodiment of the invention, the infection that is treated according to the invention is mediated by an organism that requires glycogen, or glucose that results from the breakdown of glycogen, as a source of energy and/or carbon supply.

The terms “treating”, “treat”, “treatment”, as used herein, includes curative, preventative (e.g. prophylactic) and palliative treatment.

The term glycogen phosphorylase inhibitor refers to a compound or agent which reduces, retards or eliminates the enzymatic action of glycogen phosphorylase. The currently known enzymatic action of glycogen phosphorylase is the degradation of glycogen by catalysis of the reversible reaction of a glycogen macromolecule and inorganic phosphate to glucose-1-phosphate and a glycogen macromolecule which is one glucosyl residue shorter than the original glycogen macromolecule (forward direction of glycogenolysis).

Administration of the glycogen phosphorylase inhibitor and azithromycin can be via any method which delivers a compound of the combination of this invention systemically and/or locally. These methods include oral routes, parenteral, intraduodenal routes, et al. Generally, the compounds used in this invention are administered orally, but parenteral administration (e.g., intravenous, intramuscular, transcutaneous, subcutaneious or intramedullary) may be utilized, for example, where oral administration is inappropriate for treating the infection or related disease, or where patient is unable to ingest the drug. Topical administration may be indicated when the medication is best applied to the surface of a tissue or organ as determined by the attending physician.

The glycogen phosphorylase inhibitor and antibiotic can be separately co-administered simultaneously or sequentially in any order, or at different times. Alternatively, a single pharmaceutical composition comprising effective amounts of azithromycin and a glycogen phosphorylase inhibitor disclosed herein can be administered.

In any event, the amount and timing of administration of the azithromycin and glycogen phosphorylase inhibitor will, of course, be dependent on the subject being treated, on the severity of the affliction, on the manner of administration and on the judgment of the prescribing physician. Thus, because of patient to patient variability, the dosages given below are a guideline and the physician may titrate doses of the drug to achieve the activity (e.g., antibacterial and/or antiprotozoan activity) that the physician considers appropriate for the individual patient. In considering the degree of activity desired, the physician must balance a variety of factors such as age and weight of the patient and the presence of other diseases. The following paragraphs provide preferred dosage ranges for the various components of this invention.

An effective dosage for the glycogen phosphorylase inhibitor is from about 0.7 to about 7,000 mg per day. For a normal adult human having a body weight of about 70 kg, dosage in the range of about 0.01 to about 100 mg per kilogram of body weight is typically sufficient.

An effective dosage for azithromycin is generally from about 250 mg to about 500 mg/day for five days, or a single dose of from 1 to 2 grams. Dosage forms and amounts of azithromycin are well known and can be easily determined by the treating physician.

The determination of dosage ranges and optimal dosages for a particular patient is well within the ordinary skill in the art in light of this disclosure.

The antibiotic and glycogen phosphorylase inhibitor are generally administered in the form of a pharmaceutical composition comprising at least one of the compounds of this invention together with a pharmaceutically acceptable vehicle or diluent. Thus, the compounds of this invention can be administered individually or together with any conventional oral, parenteral or transdermal dosage form.

When administered together in a dosage form, an additional aspect of the invention provides pharmaceutical compositions comprising azithromycin and a glycogen phosphorylase inhibitor, or a pharmaceutically acceptable salt thereof or prodrug thereof. Preferably, the pharmaceutical compositions contain sufficient amounts of antibiotic and glycogen phosphorylase inhibitor such that, after one or more doses of the pharmaceutical composition, synergistic effective amounts of the antibiotic and glycogen phosphorylase inhibitor are present in the patient.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluable salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples, see Remington's Pharmaceutical Sciences, Mack Publishing Company, Easter, Pa., 15^{th} Edition (1995).

Pharmaceutical compositions according to the invention may contain 0.1%95% of the antibiotic and glycogen phosphorylase inhibitor, preferably 1%-70%. In any event, the composition or forumulation to be administered will contain sufficient antibiotic and glycogen phosphorylase inhibitor such that, after one or more doses of the pharmaceutical composition, effective amounts of the antibiotic and glycogen phosphorylase inhibitor in combination are present in the patient.

Pharmaceutically acceptable salts and prodrugs of the glycogen phosphorylase inhibitors and azithromycin are within the scope of the methods, pharmaceutical compositions and kits of the invention. The term pharmaceutically acceptable salts and prodrugs refers to the salts, amino acid addition salts and prodrugs of a glycogen phosphorylase inhibitor or antibiotic that are, within the scope of sound medical judgment, suitable for use with patients without undue toxicity, irritation, allergic response, and the like, commensurate with a reasonable benefit/risk ratio, and effective for their intended use.

The term “salts” refers to inorganic and organic salts of the glycogen phosphorylase inhibitor or antibiotic. The salts can be prepared in situ during the final isolation and purification of a compound, or by separately reacting a compound with a suitable organic or inorganic acid and isolating the salt thus formed. Representative salts include hydrobromide, hydrochloride, sulfate, bisulfate, nitrate, acetate, oxalate, palmitate, stearate, laurate, borate, benzoate, lactate, phosphate, tosylate, citrate, besylate, maleate, fumarate, succinate, tartrate, naphthylate, mesylate, glucoheptonate, lactobionate, and laurylsulphonate salts, and the like. The salts may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, for example, S. M. Berge, et al., “Pharmaceutical Salts,” J Pharm Sci, 66:1-19 (1977).

The term “prodrug” means a compound that is transformed in vivo to yield a glycogen phosphorylase inhibitor. The transformation may occur by various mechanisms, such as through hydrolysis in blood. Suitable prodrugs include the prodrugs disclosed in U.S. Pat. Nos. 6,107,329 and 6,297,269. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

The glycogen phosphorylase inhibitor or antibiotic may exist in solvated and hydrated forms. The present invention encompasses both the solvated and hydrated forms of the glycogen phosphorylase inhibitor and antibiotic as well as the unsolvated and non-hydrated forms.

Since one aspect of the present invention contemplates the treatment of infection or atherosclerosis with a combination of azithromycin and glycogen phosphorylase inhibitor that may be administered separately in any order, the invention further relates to combining separate pharmaceutical compositions in kit form. The kit comprises two separate pharmaceutical compositions: a glycogen phosphorylase inhibitor as disclosed herein, and azithromycin. The kit further comprises a container for containing the separate compositions such as a divided bottle or a divided foil packet. Additional examples of containers include syringes, boxes, bags, and the like. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil, which is opposite from the direction in which the recesses are formed. As a result, the tablets or capsules are sealed in the recesses between the plastic foil and the sheet. Preferably, the strength of the sheet is such that the tablets or capsules can be removed from the blister pack by manually applying pressure on the recesses whereby an opening is formed in the sheet at the place of the recess. The tablet or capsule can then be removed via the opening.

The invention additionally relates to kits comprising azithromycin in a unit dosage form and instructions for administering a glycogen phosphorylase inhibitor to a mammal, and kits comprising a glycogen phosphorylase inhibitor in a unit dosage form and instructions for administering azithromycin to a mammal. The instructions for administering the glycogen phosphorylase inhibitor or azithromycin to a mammal will include instructions for administering such compounds in accordance with the present invention.

### EXAMPLE

Antibacterial effect of azithromycin in combination with 5-chloro-1H-indole-2-carboxylic acid [(1S)-(4-fluoro-benzyl)-2-(4-hydroxypiperidin-1-yl)-2-oxo-ethyl]-amide (referred to as Compound A) or 5-chloro-1H-indole-carboxylic acid [(1S) benzyl-3-((3R,4S)-dihydroxy-pyrrolin-1-yl)-(2R)-hydroxy-3-oxo-propyl] amide (referred to as Compound B).

Hep2 cells maintained in Isocove's Modified Dulbecco's Medium (IMDM) were infected with Chlamydia pneumonia (0.5 MOI for two hours after one hour centrifugation). Cells were then treated with Compound A (0, 5, 10, 20, 30 or 40 μg/ml) or Compound B (0, 5, 10, 20, 30 or 40 μg/ml) alone and/or in combination with azithromycin (Pfizer, Inc., New York, N.Y.) 0, 0.005, 0.0158, 0.050, 0.158, or 0.5 μg/ml). For minimum inhibitory concentration (MIC) determination, cells were terminated for analysis at 72 h after the addition of drugs. For minimum bacteriocidal concentration (MBC) determination, drugs were removed at 72 h post-treatment, the media was replaced, and cells were maintained for an additional 72 h before termination. At termination, cells were analyzed for quantitation of inclusion bodies using fluorescent microscopy, and general cell viability by microscopic visualization.

Azithromycin alone had an MIC of 0.158 μg/ml and an MBC of 0.5 μg/ml.

Compound A Compound A alone had an MIC of 20 μg/ml and an MBC of 30 μg/ml. For the combination of azithromycin and Compound A, there was a 3-fold decrease in azithromycin concentration required for MIC (0.05 μg/ml) when combined with 5 μg/ml Compound A. There was a 10-fold decrease in azithromycin concentration required for MIC (0.0158 μg/ml) when combined with 10 μg/ml Compound A. There was a 3-fold decrease in azithromycin concentration required for MBC (0.158 μg/ml) when combined with 5 μg/ml Compound A. There was a 10-fold decrease in azithromycin concentration required for MBC (0.05 μg/ml) when combined with 20 μg/ml Compound A. The concentrations of Compound A required to reduce the MIC or MBC for azithromycin were significantly lower than concentrations associated with changes in host cell appearance (e.g. 30+μg/ml).

Compound B The MIC and MBC of Compound B were both greater than the highest concentration tested, e.g. 40 μg/ml. For the combination of azithromycin and Compound B, there was a 3-fold decrease in azithromycin concentration required for MIC (0.05 μg/ml) when combined with 10 μg/ml Compound B; a 10-fold decrease in azithromycin concentration required for MIC (0.0158 μg/ml) when combined with 30 μg/ml Compound B; and a 3-fold decrease in azithromycin concentration required for MBC (0.158 μg/ml) when combined with 30 μg/ml Compound B. The concentrations of Compound B required to reduce the MIC or MBC for azithromycin were not associated with any changes in host cell appearance.

The results show a synergy of antibacterial effects by the combination of Compound A and azithromycin, and Compound B and azithromycin.

1. A method of treating a bacterial infection in a mammal comprising administering to a mammal in need of such treatment effective amounts of azithromycin and a glycogen phosphorylase inhibitor.

2. The method of claim 1 wherein said glycogen phosphorylase inhibitor is selected from the group consisting of 5-chloro-1H-indole-2-carboxylic acid [(1S)-(4-fluorobenzyl)-2-(4-hydroxypiperidin-1-yl)-2-oxoetheyl]amide and 5-chloro-1H-indole-2-carboxylic acid [(1S)-benzyl-3-((3R,4S)-dihydroxypyrrolidin-1-yl)-(2R)-hydroxy-3-oxopropyl]amide.

3. The method of claim 1 wherein said bacterial infection is a Chlamydia pneumoniae infection.

4. A method of treating a Chlamydia pneumoniae infection comprising administering to a mammal comprising administering to a mammal in need of such treatment effective amounts of azithromycin and a glycogen phosphorylase inhibitor.

5. The method of claim 4 wherein said glycogen phosphorylase inhibitor is selected from the group consisting of 5-chloro-1H-indole-2-carboxylic acid [(1S)-(4-fluorobenzyl)-2-(4-hydroxypiperidin-1-yl)-2-oxoethyl]amide and 5-chloro-1H-indole-2-carboxylic acid [(1S)-benzyl-3-((3R,4S)-dihydroxypyrrolidin-1-yl)-(2R)-hydroxy-3-oxopropyl]amide.

6. A method of treating atherosclerosis comprising administering to a mammal in need of such treatment effective amounts of azithromycin and a glycogen phosphorylase inhibitor or a pharmaceutically acceptable salt thereof or prodrug thereof.

7. The method of claim 6 wherein said glycogen phosphorylase inhibitor is selected from the group consisting of 5-chloro-1H-indole-2-carboxylic acid [(1S)-(4-fluorobenzyl)-2-(hydroxypiperidin-1-yl)-2-oxoethyl]amide and 5-chloro-1H-indole-2-carboxylic acid [(1S)-benzyl-3-((3R,4S)-dihydroxypyrrolidin-1-yl)-(2R)-hydroxy-3-oxopropyl]amide.

8. A pharmaceutical composition comprising, in effective amounts, azithromycin and a glycogen phosphorylase inhibitor or a pharmaceutically acceptable salt thereof and further comprising a pharmaceutical carrier or diluent.

9. A pharmaceutical composition of claim 8 wherein said glycogen phosphorylase is selected from the group consisting of 5-chloro-1H-indole-2-carboxylic acid [(1S)-(4-fluorobenzyl)-2-(4-hydroxypiperidin-1-yl)-2-oxoethyl]amide and 5-chloro-1H-indole-2-carboxylic acid [(1S)-benzyl-3-((3R,4S)-dihydroxypyrrolidin-1-yl)-(2R)-hydroxy-3-oxopropyl]amide.

10. A kit comprising:
a) azithromycin or a pharmaceutically acceptable salt thereof and a pharmaceutically acceptable carrier or diluent in a first unit dosage form;
b) a glycogen phosphorylase inhibitor or a pharmaceutically acceptable salt thereof and a pharmaceutically acceptable carrier or diluent in a second unit dosage form; and
c) a container.

11. The kit of claim 10 wherein said glycogen phosphorylase inhibitor is selected from the group consisting of 5-chloro-1H-indole-2-carboxylic acid [(1S)-(4-fluorobenzyl)-2-(4-hydroxypiperidin-1-yl)-2-oxoethyl]amide and 5-chloro-1H-indole-2-carboxylic acid [(1S)-benzyl-3-((3R,4S)-dihydroxypyrrolidin-1-yl)-(2R)-hydroxy-3-oxopropyl]amide.

12. A kit comprising:
a) azithromycin or a pharmaceutically acceptable salt thereof and a pharmaceutically acceptable carrier or diluent in a unit dosage form; and
b) instructions for administering a glycogen phosphorylase to a mammal.

13. The kit of claim 12 wherein said glycogen phosphorylase inhibitor is selected from the group consisting of 5-chloro-1H-indole-2-carboxylic acid [(1S)-(4-fluorobenzyl)-2-(4-hydroxypiperidin-1-yl)-2-oxoethyl]amide and 5-chloro-1H-indole-2-carboxylic acid [(1S)-benzyl-3-((3R,4S)-dihydroxypyrrolidin-1-yl)-(2R)-hydroxy-3-oxopropyl]amide.

14. A kit comprising:
a) a glycogen phosphorylase inhibitor or a pharmaceutically acceptable salt thereof and a pharmaceutically acceptable carrier or diluent in a unit dosage form; and
b) instructions for administering azithromycin to a mammal.

15. The kit of claim 14 wherein said glycogen phosphorylase inhibitor is selected from the group consisting of 5-chloro-1H-indole-2-carboxylic acid [(1S)-(4-fluorobenzyl)-2-(4-hydroxypiperidin-1-yl)-2-oxoethyl]amide and 5-chloro-1H-indole-2-carboxylic acid [(1S)-benzyl-3-((3R,4S)-dihydroxypyrrolidin-1-yl)-(2R)-hydroxy-3-oxopropyl]amide.
